(19) United States
(12) Patent Application Publication
(10) Pub. No.: US 2010/0003760 A1
(43) Pub. Date: Jan. 7, 2010

(54) Analysis of beryllium in soils and other samples by fluorescence

(75) Inventors: Anoop Agrawal, Tucson, AZ (US); John P. Cronin, Tucson, AZ (US); Juan Carlos Tonazzi, Tucson, AZ (US)

Correspondence Address: LAWRENCE R. OREMLAND, P.C., 5055 E. BROADWAY BLVD., SUITE C-214, TUCSON, AZ, 85711, US

(73) Assignee: AJJER LLC, Tucson, AZ (US)

(21) Appl. No.: 12/040,808

(22) Filed: Feb. 29, 2008

Related U.S. Application Data
(60) Provisional application No. 60/904,513, filed on Mar. 1, 2007.
(60) Provisional application No. 60/919,584, filed on Mar. 23, 2007.

(51) Int. Cl. G01N 33/20; G01N 33/24

(52) U.S. Cl. 436/27; 436/79; 436/172

(57) ABSTRACT

An improved low-cost practical method of determining beryllium or a beryllium compound thereof in a sample is disclosed by measuring fluorescence. This method discloses methods to lower the back ground fluorescence. Further, the method is extended to improved analysis of beryllium in soils by including a heating step.

### RELATED APPLICATION/CLAIM OF PRIORITY

This application is related to and claims priority from provisional application 60/904,513 entitled Improved Methods to Analyze Beryllium by Fluorescence filed on Mar. 1, 2007; and provisional application 60/919,584 entitled Methods to Reduce Background in Analysis of Beryllium by Fluorescence filed on Mar. 23, 2007 which are all incorporated by reference herein.

### UNITED STATES GOVERNMENT RIGHTS

This invention was made with US Government support under contract number DE-FG02-06ER84587 awarded by the Department of Energy. The Government has certain rights to this invention.

### FIELD OF THE INVENTION

The present invention relates to the detection and quantification of beryllium by fluorescence. More particularly, the present invention relates to high sensitivity detection and quantification of beryllium in air, water, surfaces, or bulk samples (for example soils).

### BACKGROUND OF THE INVENTION

Beryllium is a metal that is used in a wide variety of industries including electronics, aerospace, defense, and the Department of Energy (DOE) complexes. Exposure to beryllium containing particles can lead to a lung disease called Chronic Beryllium Disease (CBD). CBD involves an uncontrolled immune response in the lungs that can lead to deterioration in breathing capacity and ultimately death. It is clear that even in processes where beryllium dust has been controlled to very low levels, cases of disease still persist. In fact, there have been cases of CBD reported in people that have had no obvious direct contact with beryllium operations. Despite the fact that very low exposure levels can lead to CBD, the onset of disease can take decades.

Recent new regulations from DOE dictate a permissible exposure limit of 0.2 μg/m³ in air, a housekeeping level of 3 μg/100 cm² on a surface, and a release level for materials after beryllium exposure where the surface contamination due to beryllium must not exceed 0.2 μg/100 cm².

There is a discussion in the beryllium community that the permissible air exposure limit of beryllium needs to be lowered to 0.02 μg/m³. Currently, thousands of surface wipes and air filters are analyzed annually for beryllium. In addition OSHA has detected airborne levels of beryllium at numerous sites within the United States. In some of the sites where past beryllium activity or disposal has taken place, beryllium needs to be cleaned from the soil, down to a level of 131 mg of beryllium in each kg of soil. The present technique for detecting beryllium is a surface analysis which involves wiping an area with a filter paper, performing a microwave digestion with acid to dissolute beryllium or its compounds, and then analyze by inductively coupled plasma (ICP) atomic emission spectroscopy (AES). For analyzing airborne samples, one draws a known quantity of air through a filtering medium and then the filter is treated in a similar fashion to the surface wipes. The ICP-AES technique also requires highly trained operators and the entire sample is consumed during the analysis, so that a sample that is identified as positive for beryllium cannot be checked or verified with a second run. In addition, since there are many elements present in soils, there is always an issue with interference amongst the various elements in order to accurately quantify beryllium.

Although there are several reports of being able to detect beryllium with a fluorescent indicator (see Matsumiya), only recently quantitative fluorometric beryllium detection methods have been shown to be effective for the current exposure regulations. Three key elements to a useful detection system that have been missing previously are: first, the detection system must be capable of dissolving both beryllium oxide and beryllium metal; second, the detection system must work in the presence of other metals and fluoride ions: third, the detection system must be easy to use and preferably offer the ability to be used in the field. Most fluorescent indicators reported in the literature do not tolerate the presence of fluoride ions, which is critical if a fluoride-based medium is used to dissolve the beryllium. The few reports of fluorescent indicators that can tolerate fluorides, have used complicated procedures involving heating with acid for dissolution and a titration process to obtain the final pH that require long periods of time and prohibit use in the field.

The extensive chemistry required in previous fluorescent systems and interferences from other metals have limited their use, and to date there is no simple approach to beryllium detection by fluorescence. A quick, simple and specific approach has now been developed for the detection and quantification of beryllium as claimed in U.S. Pat. No. 7,129,093 which is incorporated herein by reference. This method is specific to beryllium and there are no interferences caused by other elements. Further this method provides a quantitative method of determining beryllium or a compound thereof (including beryllium oxide) in a sample, which has a fast turnaround time and can be made to be readily field portable. Moreover, the method disclosed in U.S. Pat. No. 7,129,093 has been further developed by the method and kit disclosed in U.S. patent application Ser. No. 11/152,620 filed on Jun. 14, 2005 entitled Method and Kits to Detect Beryllium by Fluorescence (which claims priority to Provisional Application Ser. No. 60/581,234, filed Jun. 18, 2004), which is also incorporated by reference herein.

One object of the present invention is to demonstrate practical methods of analyzing soils for beryllium by fluorescence or wipe and filter samples heavily contaminated by dust.

Another objective of this invention is to increase the sensitivity of the test by decreasing the background signal during fluorescence measurement.

Yet another objective of the invention is to analyze beryllium in air by drawing it through a liquid medium.

### SUMMARY OF THE INVENTION

In accordance with the purposes of the present invention, as embodied and broadly described herein, the invention provides a method of determining the presence and amount of beryllium or a beryllium compound in a sample including admixing a sample suspected of containing beryllium or a beryllium compound with a dissolution solution for sufficient time whereby beryllium or a beryllium compound within said sample is dissolved, mixing a portion from the admixture with a buffered solution containing a fluorescent indicator capable of binding beryllium or a beryllium compound to the fluorescent indicator, and, determining the presence of an amount of beryllium or a beryllium compound within the sample by measuring fluorescence from the fluorescent indicator. For practical kits, it is important that the dissolution solutions and the buffered detection solutions have a long shelf life so that these may be easily transported and stored for a length of time without deterioration or loss of their properties.

Further, it is preferred that a low cost instrument be used to detect the beryllium by fluorescence. It is further preferred that such an instrument be portable. It has been found that with proper selection of optical filters on these instruments, the low cost detectors employing photomultiplier tubes and photosensors may be used for detection of fluorescence signals yielding sensitivity down to less than 1 part per billion, and possibly below 100 parts per trillion.

Particulates of beryllium and its compounds may be collected by wiping a surface suspected of being covered with them or by capturing particles on a filter as the air is passed through it. Alternatively, beryllium may be monitored in the air by separating and collecting beryllium particles by passing the air over a series of meshes with decreasing mesh size and then analyzing the separated samples for beryllium. In both cases the wipe or the filter is first treated in the dissolution solution to extract beryllium. Particularly for air sampling, the beryllium particles may be separated based on their size and collected so that their analysis may yield a size distribution. In an alternative method, the air with beryllium particulates may be passed through a liquid media which traps the particulates and then the liquid media is analyzed manually or automatically. In addition, soils, sedimentations, fly ash, dust, crushed rocks and sands (called soils collectively) that may have been contaminated with beryllium and its compounds (e.g., beryllium oxide, beryllium acetate, beryllium sulfate) may also be collected and analyzed for total amount of beryllium including that which may be naturally present in these materials. Prior to analysis, the soil samples may have to be further prepared using standard soil preparation protocols which include, drying, milling and sieving to ensure consistency.

### BRIEF DESCRIPTION OF THE DRAWINGS

FIG. 1: Absorption spectra of freshly prepared detection solution, with and without beryllium;

FIG. 2: Fluorescence spectra of detection solution with various concentrations of beryllium;

FIG. 3: Schematics of the optical layout of the fluorescence measurement system;

FIG. 4: Effect on fluorescence signal of a sample with changing temperature;

FIG. 5: Effect of standing time (of measurement solution) on absorbance spectra of the NIST SRM Marine sediment;

FIG. 6: Schematics of optical layout to reduce background from the sample.

### DETAILED DESCRIPTION

Typically, 1250 to 2000 liters of air at a flow-rate of 1 to 4 liters/minute is used to collect particulates on the media. As an example, the DOE regulations (10CFR850) state that airborne beryllium in work space must be less than 0.2 μg/m³, which is generally measured by personal samplers (carried by workers in beryllium contaminated area) over an eight hour shift. This is a time weighted average (TWA), where the air is sampled over an eight hour shift and the filter from the sample is then analyzed. Since one cubic meter comprises of 1000 liters, this will require a pump drawing about 2.5 liter of air per minute in order to draw sufficient air over the entire shift in order to see if the contamination is above or below the regulatory limit. The reason one is not able to collect smaller volumes of air using a smaller more convenient pump, is because the most common current technology utilizing ICP-AES is only able to quantify reliably to this limit. It would be highly desirable if a smaller pump (thus lighter samplers) can be used by workers who are employed for long periods in such environments. As an example, if the technology is provided that is reliably able to measure 0.02 μg of beryllium, then one needs to only draw 0.1 m³ of air and a lighter personal pump will suffice. This is a very important benefit to recognize, i.e., the ability to go to finer detection limit enables one to measure workers for short exposure periods to see if they were exposed to equivalent of the existing code that is to DOE 10CFR850 at 0.2 μg/m³ of exposure over eight hours. As an example, a personal sampling pump which is generally used by beryllium workers is model Aircheck 2000 (supplied by SKC Inc located in Eighty Four, Pa.). This is able to draw up to 3.25 liters/minute of air and weighs 22 ounces (624 g). Another model “pocket pump” available from the same company is able to draw up to 0.225 liter/minute of air and only weighs 5 ounces (or 142 g). Thus the “pocket pump” can be used reliably to protect workers while relieving them of the burden of carrying a heavy sampler. Alternatively, one may look at this issue in another way. What happens if a worker is exposed to a high dose of a beryllium plume over a short period? Could the larger air sampler be used to collect smaller amounts of air over a shorter period so that the filters could be analyzed more frequently? The ability to measure down to lower limits (so that air or sample size is small) enables this choice as well. Thus it is desirable to sample 100 liter or less of air to be able to measure 0.2 μg of beryllium per cubic meter of air, or alternatively use personal air samples that draw less than 1 liter of air/minute, and more preferably less than 0.5 liter of air/minute. Further, it is highly advantageous to be able to measure this using fluorescence. Since the instruments can be made portable and are low in capital cost, a number of these can be used within the sites rather than transporting samples to a central location which can take time and added expense. In many situations equipment is used to conduct experiments in beryllium contaminated area. This equipment is expensive and needs to be hauled to another site, however, until the analytical results are obtained from surface wipes certifying that the equipment is clean, it cannot be removed from a beryllium contaminated area to a non-contaminated area. This leads to loss of productivity and the instruments are kept away from users for several days or weeks. Further, as ammonium bifluoride (ABF) solutions for dissolution are less toxic than many of the concentrated acids used in the industry, it is easier to manage waste. Thus, it is very desirable to use ABF solutions for dissolution and being able to measure 0.02 μg (or lower) of beryllium per cubic meter of air, or alternatively use personal air samplers that draw less than Iliter of air/minute, and more preferably less than 0.5 liter of air/minute to measure up to the regulations (e.g., 0.2 μg/m³ as called by DOE) within a standard work shift, i.e., within eight hours or less.

The media or the filter which has trapped beryllium particulates are analyzed for quantification. The air-sampling device may be a portable one being worn by a person or it may be mounted in a specific work area. Particularly for air sampling, the beryllium particles may be separated based on their size so that their distribution may be determined. Any method may be used to collect and separate beryllium particles, for example, air is drawn through a series of meshes with decreasing mesh size. Beryllium particles, if any, are thus separated based on their size and then collected. This collection may be on a media, such as a porous or filter paper or cloth which will capture these particles. Each of the fractions are then analyzed separately for beryllium quantification to obtain a distribution of mass of beryllium vs. particle size. It may be important to analyze different work places for particle sizes, as it may provide important information on why some work places result in higher number of CBD cases. For example, 50% cut-off for the size of respirable particles (aerodynamic diameter) is about 4 microns, with almost 10 micron sized particles entering the respiration glands. For thoracic glands the 50% cut-off is at 10 microns with almost up to 30 micron particles entering this area.

Generally, large air pumps are located in the beryllium workplaces which pull the air through the filters and analyzed periodically. This is labor intensive, as filters have to be removed and then taken to an analytical lab. An alternate is to draw the air through a liquid medium. Preferably the liquid medium also acts as a dissolution solution for the toxic particulates, which in this case are beryllium and its compounds. Thus ammonium biflouride aqueous solution (typically in concentrations of 10 g/liter to 50 g/liter) may be used for this purpose. As the particles are pulled through the liquid, the dissolution process begins. The liquid reservoir may be removed manually for further analysis while it is replaced with another one, the liquid may be siphoned or replaced automatically at pre-set intervals or if an event is triggered. This event could be a sudden change in fluid property, e.g. fluoride content, pH, color or any other property, or if one opens a door to a contaminated chamber or it is suspected that accidentally a plume of beryllium particulates may have been released. One may wait for some time for dissolution to be complete for the last of the particles that enter the liquid reservoir, or one may analyze the liquid immediately and also after some time (e.g., 30 minutes) and compare the results. As an example an air sampler that may be used is called Omni 3000 (from Sceptor Industries, Kansas City, Mo.). This has an adjustable air flow up to 300 liter/minute which is directed via a removable liquid cartridge. For example, at 250 liter/minute, it can draw one cubic meter of air in four minutes. Thus, one may be able to sample the air and analyze this at five minute intervals. This may be done in a sampling time of less than 1 minute using finer detection limits as explained earlier. This does not include the dissolution time, thus the results would be delayed by the time taken for complete dissolution and further sample processing. The liquid level in the cartridge may drop as the air leaving the cartridge may carry some moisture. But this is automatically adjusted in this unit by adding make up water or the dissolution fluid as originally contained in the cartridge. This unit may be further synchronized with an automated analyzer so that the work place can be monitored continuously and sounds an alarm when the readings exceed an accepted level. There is enough agitation in the cartridge due to the air flow, but one may also employ a heater to keep the liquid at an elevated temperature. Typically it has been shown that beryllium oxide dissolutes faster in ammonium bifluoride at elevated temperature. Preferred temperature is usually in the range of 50 to 100 C, but a more preferred temperature range is 70 to 90 C.

A variety of wipes and wiping methods may be used. For example ASTM D6966 describes methods on how to wipe in order to collect the particles efficiently. One may use dry wipes or wet wipes. Dry wipes may work better on softer surfaces as compared to the harder ones. The wetting medium for wet wipes may be aqueous or non-aqueous. Aqueous medium may have surfactants to change the surface tension in order to wet and capture the particles more efficiently. Surfactants may be ionic or non-ionic. Some of the surfactants are polyethylene and polypropylene glycols in various molecular weights as Triton™ available from Aldrich Chemical Company (Milwaukee, Wis.). Some examples of Triton™ are N-101 reduced, SP-135, SP-190, X-100, X-100 reduced, X-114, X-114 reduced, X-405 and X-405 reduced. Usually, the molecular weight of the glycols for this purpose is lower than 5000 and preferably lower than 2000. Since these materials have high molecular weight, their vapor pressure is lower as compared to water, thus they do not have a tendency to dry out and may be used by themselves as the wetting media. Non-drying wetting fluids can leave stains on the surfaces which may take long to dry or require a clean-up later. The most preferred wetting media is water, or water comprising surfactants.

The wipe may comprise paper, fabrics, felts and filters, comprising cellulose, cellulose esters, nitrocellulose, acrylic, polyvinyl acetate, nylon, polyvinyl alcohol, polyester, polycarbonate, polytetrafluoroethylene, polyvinylidene fluoride, polyolefins, natural fibers (e.g., cotton, jute, hemp, wool, silk, hair) or any other media which serves the purpose of collection, easily releases captured particles in the dissolution solution and preferably does not disintegrate in the dissolution solution. These may be hydrophilic or hydrophobic. To increase the efficiency of collection from dry or wet wipes their surfaces may be engineered so that pores are provided on their surfaces in the same size range as the expected particle sizes so as to firmly collect and lodge the particles. An example of such engineered surfaces may be filters made out of various materials (e.g. see 2005 Catalog from Fisher Scientific page 518 to page 529 (Pittsburgh, Pa.), or for example StretchN'Dust® from Chicopee (Mooresville, S.C.)). Another example of these are ashless paper filters from Whatman (Haverhill, Mass.) type 541. Further it is preferred that the media is wetted before collecting the particles from the surface. It is also preferred that water in a pre-determined quantity is used for this purpose. As an example for filters 541 in a size of 47 mm in diameter (or 17.3 square cm in surface area), it has been found that wetting with about 200 microliters of water is sufficient. Generally the volume of wetting media is proportional to the surface area of the collection media, which should typically be in the range of 2 to 500 microliters of fluid per square cm of the media area. It is important that consistent amounts of wetting material be used, the surface wiped and wipe transferred to the dissolution tube solution immediately. This keeps the dilution of the dissolution solution by the wetting agent small and consistent. These media may also be used to collect samples from surfaces and air in other ways. For example micro-vacuuming may be used on a surface and particles collected on the media.

Beryllium and its compounds in liquid media such as water can be analyzed by this method. If the beryllium is present as particulates then it may be filtered using the media (or filters) as described above, and then the filter is preferably dried and put in a dissolution tube. Alternatively for solutions, one may place predetermined amount of beryllium comprising solution on to a filter or the media described above, evaporate the solvent and subject the filter to the same dissolution process.

Development of a method to analyze soils requires that the dissolution solution is able to extract added beryllium impurities (anthropogenic) and native beryllium. Soils include pulverized rocks, marine and stream sediments, fly ash and sands. Since most dissolution solutions are acids or reactive towards silicates, it is more convenient to have a method which extracts all of the beryllium from the soil matrix. One may use solutions from known methods to totally digest soils in order to analyze their compositions. Some of these methods are from Environmental Protection Agency (EPA) such as SW846-3051 and 3050 which use concentrated acid such as nitric acid which may be mixed with hydrogen peroxide and concentrated hydrochloric acid, or one may use ammonium bifluoride as it reacts with the silicates. It was surprising, that ammonium bifluoride (ABF) was quite effective in extracting beryllium from soils. We found that a preferred weight ratio of ABF to soil should be greater than 2, and a more preferred ratio was greater than 3, and most preferred ratio was greater than 4. The ABF is preferably used as an aqueous solution to which the soil is added. A preferred concentration of ABF for soil dissolution was less than 10% (10 g of ABF in 100 ml of water), and more preferably a 5% ABF solution. The preferred dissolution temperature is greater than 50 C, and more preferably in a range of 70 to 100 C and most preferable range is between 80 and 90 C. This is because the rate of attack of ammonium bifluoride on silicates increases with temperature and increasing concentration. A preferred dissolution time is greater than 60 minutes, and more preferably greater than 180 minutes. These times may be shortened by using agitation and ultrasonic vibration while being heated. A higher concentration of ABF solution may be used, but then dilution (e.g., with water or more dilute ABF solution) will be required after dissolution so that the pH of the measurement solution is still high (preferably greater than 11, and more preferably greater than 12). The work on determination of beryllium in soils by Agrawal et al, has been recently accepted for publication in Environmental Science and Technology Journal. To extract beryllium from most samples using 3% ABF solution required 40 hours at 90 C. This is a long time, but since it is a simple step of baking at 90 C, thus hundreds of samples may be placed for simultaneous processing in an oven. Since it has been shown that refractory beryllium oxide (e.g., high fired beryllium oxide such as UOX125 from Brush Welman (Cleveland, Ohio) may be dissoluted in ammonium bifluoride solution in 30 minutes at temperatures in excess of 70 C, thus the temperature, time, concentration of ABF, and the ratio of ABF to the sample may be used as a means for determination of the amount and of differentiation between the added beryllium and the natural beryllium in soils. Particularly as the dissolution of natural beryllium in soils is more difficult in terms of time, temperature or concentration of ABF solution.

The advantages of the process of the present invention include: a simple dissolution step that can dissolve particulate beryllium oxide and beryllium metal in less than thirty minutes by agitation; tolerance of a wide variety of other metals and fluoride at large concentrations; the use of a final buffered solution to avoid titration, a fast turnaround time of less than one hour and the ability to be field portable. The dissolution technique involves preferable use of ammonium bifluoride as this rapidly dissolves several beryllium compounds including beryllium metal and beryllium oxide. Further, a buffered solution including the fluorescent indicator is used and is essential for fast detection that can be done in the field. It is preferred that the concentration of ammonium bifluoride be as low as needed for dissolution so that when it is mixed with the fluorescent indicator (detection solution), the pH remains high for strong fluorescent signal. Any concentration of the ammonium bifluoride solution may be used as long as the pH of the mixture of the two solutions is basic as discussed later.

As a preferred fluorescent indicator, 10-hydroxybenzo[h]quinoline-7-sulfonate (10-HBQS) is used. The buffered solution preferably includes a buffer having a pKa between about 7 and 13.5 and more preferably in excess of 12.5. A typical buffer that is preferred is an amine buffer and most preferably is an amino acid such as lysine. Any of the lysine compounds may be used, e.g., D-lysine, L-lysine, DL-lysine, their monochlorides and dihydrochlorides. A preferred lysine compound is L-lysine monohydrochloride. The solution may also contain aminocarboxylates such as ethylenediaminetetraaceticacid (EDTA), diethylenetriaminetetraacetic acid (DTTA), triethylenetetraminehexaacetic acid (TTHA), and the like, or salts thereof, as a chelating agent to bind metals other than beryllium. Preferred salts of EDTA are dipotassium dihydrate and disodium dihydrate. Other chelating agents such as aminophosphonates may be used as well. There are a few preferable choices of indicators, all of which are based on forming six-member rings with the beryllium ion bound to a phenolate oxygen and a pyridine nitrogen. The preferred indicator is 10-HBQS.

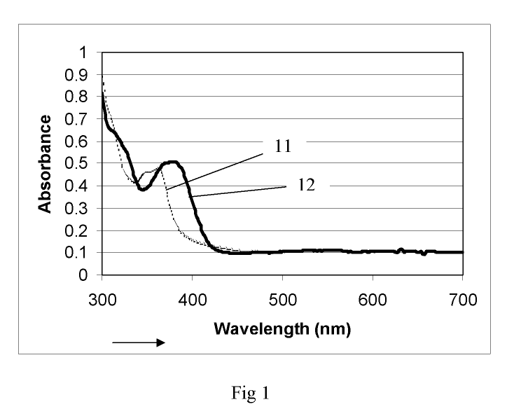

FIG. 1 shows the absorption spectra 11 of a preferred formulation of a detector solution. The solution was made by using 1.8 liters of de-ionized water (electrical resistance of water was greater than or equal tol8 Mohms), 19.51 g of lysinemonohydrochloride, 1.99 g of EDTA disodium dihydrate, 0.0367 g of 10-HBQS and then titrating this with a solution of 2.5N sodium hydroxide to a final pH of 12.85. This figure also shows the absorption spectra 12 of the same solution but after adding beryllium at 20 ppm final concentration.

The method of the present invention involves obtaining a sample on a medium (such as on a filter paper by wiping a surface or capturing airborne particles) and then placing the medium into a vial and adding 5 ml to 100 ml of an aqueous ammonium bifluoride solution for dissolution of beryllium captured on the medium. A preferred concentration is one percent ammonium bifluoride solution which can dissolve up to 10 mg of either beryllium or beryllium oxide in less than 30 minutes with simple shaking and/or heating at 80 C. A mechanical shaker or a block heater with a timer is preferred for consistency. Next, a predetermined quantity of the ammonium bifluoride solution (with dissoluted beryllium sample) is added to a buffered indicator solution after filtering (typically the filter pore size is equal to or less than 2 microns, and a preferred size is 0.45 microns), containing a fluorescent indicator and a buffer, to neutralize the solution and bind beryllium ions to the fluorescent indicator. When 10-HBQS is used as the fluorescent indicator, fluorescence at 475 nm can be used to quantitatively determine the beryllium. The most remarkable aspect of this method is its ability to tolerate a wide range of potentially interfering metals at high concentrations. A wide variety of metals including iron, aluminum, and uranium at levels 10,000 times the beryllium concentration have been reported and show no interference in detecting the beryllium (see Minogue, et al, 2005).

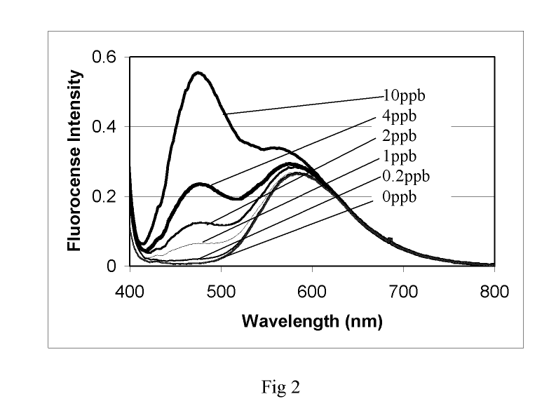

FIG. 2 shows the fluorescence spectra of the detector solution when it is mixed with various beryllium containing solutions. The peak at 475 nm is more sensitive to the beryllium concentrations. A preferred dynamic range for quantification is between 0.01 to 10 μg of beryllium on the media, and a more preferred range is between 0.001 to 20 μg of beryllium on the media. This method has high flexibility to be tailored to any desired range. If higher amounts of beryllium are suspected that go beyond the instrument range, one always has the option to dilute the solutions or to use filters to lower the excitation or the emission intensity. For soils a preferred range is from about 0.1 μg of beryllium/g of soil to about 800 μg of beryllium/g of soil, a more preferred range being from about 1 μg of beryllium/g of soil to about 200 μg of beryllium/g of soil.

In the prior art, the media is usually a filter paper (e.g., Whatman 541 for wipe and Mixed cellulose ester (MCE) filter for air sampling (see Minogue; Ashley (2005) and U.S. Pat. No. 7,129,093) spiked with different amounts of beryllium compound and dissoluted with 5 ml of 1% ammonium bifluoride solution by mechanical agitation. A 0.1 ml of this solution was added to 1.9 ml of the preferred detection solution (described above in FIG. 1) and then measured by fluorescence. Currently, this method determines between 0.014 μg and 4 μg per wipe or filter (media). This method is adequate to meet regulation standards where between 0.2 and 4 μg needs to be measured on a media (or a filter paper). Further, this method has the ability to verify a result by rerunning fluorescence or doing inductively coupled plasma atomic emission on the 4.9 ml of the dissolution solution that remains unused. However, if the regulations are changed in future to be able to reliably measure down to 0.02 μg on the media, then it would be preferred that the method detection limit is about 0.002 μg.

To increase the solubility kinetics of larger particles, particularly more refractive materials such as beryllium oxide, the dissolution solution may also comprise of acids and their mixtures, and acids mixed with ammonium bifluoride. One has to be careful that when the detection solution is mixed with the dissolution solution, the volumes used and the buffer capacity of the detection solution is such so that a high pH is maintained for the mixture when the fluorescence measurement is done. Typically pH of the mixture is preferably in excess of 10 and more preferably in excess of 12. If the pH drops, the dissolution solution (after extraction of beryllium) could be diluted by water or a more dilute ABF solution before mixing with the dye solution. Some preferred acids are hydrochloric acid, sulfuric acid, hydrofluoric acid and nitric acid. Some of the preferred acid containing dissolution solutions are made of 1% acid solutions in water to which ammonium bifluoride (ABF) is added so as to result in a final ABF concentration of 1%, for example 1% ammonium bifluoride solution (weight: volume) in 1% hydrochloric acid solution. Further the dissolution process of beryllium and its compounds captured onto the wipe in these solutions is aided by mechanical shaking and/or agitation. One may also use heat, microwaves and ultrasonic vibrations to expedite or accelerate the process. Typically the preferred temperatures are lower than 100° C., e.g., 75° C., the preferred microwave frequencies are 915 MHz and 2450 MHz and the preferred ultrasonic frequencies are in the range of 18 kHz to 300 kHz. The dissolution time for a fixed chemistry depends on the composition of the dissolution solution and the particles, particle size (e.g., surface area) and the type of acceleration factor chosen as listed above. It is desirable to select the shortest period for dissolution, preferably less than 240 minutes to ensure the results are available on the same day. For soil samples to determine their natural beryllium content the time period may be considerably longer.

The advantages of fluorescence method include a fast turnaround time and/or simple dissolution protocol, and the ability to verify a result by rerunning fluorescence or doing inductively coupled plasma atomic emission on the dissolution solution that remains unused. There are several commercial, portable fluorometers that could be used in the field. The present method from dissolution to detection is field portable, has a low detection limit, and can tolerate a wide variety of interferences. The method has the potential to save both man-hours and costs. As an example, a compact fluorometer for use in the field or a laboratory is the Modulus 9200 from Turner Biosystems (Sunnyvale, Calif.) which may be configured to run on a battery pack or automobile 12 to 24V outlet. To keep the power consumption low, this uses a 365 nm light emitting diode as excitation source. A preferred emission filter has a peak transmission in a range of 475 to 480 nm with a bandpass of less than ±20 nm. These types of instruments may also be controlled by or attached for data acquisition to a laptop or a hand held computer or personal digital assistants e.g. IPAQ (from Hewlett Packard, Palo Alto, Calif.).

To increase detection limit (meaning to be able to detect lower quantities of beryllium) the prior art method can be modified in several novel ways. As discussed below, one approach is to modify instrumentation and the other to modify the chemistry, or both for maximum impact.

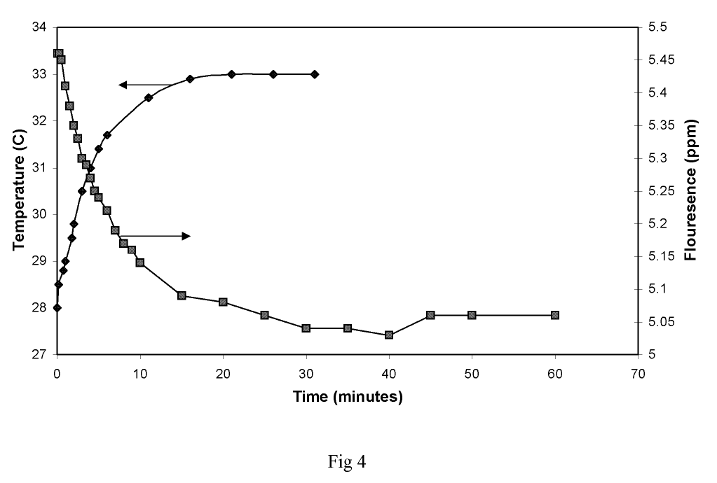

The sensitivity or the detection limit of this test can be easily increased by a factor of 10 or more, since the other metals do not interfere with the results and the test is specific to beryllium. To obtain high sensitivity and low noise in the measurement, it is important to control temperature of the solution (mixture of the dissolution solution and that of the detection solution also called “measurement solution”) while measuring fluorescence. FIG. 4 shows the change in fluorescence with temperature. This temperature must be controlled within a narrow band as compared to the temperature at which the measurements were made on mixtures of known quantities of beryllium in the detection solution or “calibration standard solutions”. In addition, a preferred range of temperature to measure fluorescence is between 10° C. and 40° C., and a more preferred range is between 10° C. and 25° C. A preferred spread of temperatures within the above mentioned range where all the standards and the sample must be analyzed is dependent on the precision required. Generally this should be within ±3° C. and more preferably within ±1° C. This means that the temperature of all calibration solutions and the samples measured against a calibration curve from these solutions should be kept within this narrow range during measurement. For low noise high sensitivity detection it is preferred to keep a tight control on the temperature. This may be done by increasing the airflow around the sample compartment as long as the air temperature in the room is strictly maintained. Another way is to have a constant temperature fluid circulation bath, or even having the temperature be controlled using Joule-Thompson or Peltier (or also called thermoelectric) devices in close proximity to the sample holder. Generally the thermoelectric (TEC) devices comprise of two ceramic plates that are separated by n-type and p-type semiconductor material. By applying an appropriate voltage to the semiconducting material it is possible to transfer heat from one of the ceramic plates to the other plate, thus creating a hot plate and a cold plate.

As discussed earlier one of the most important aspect of the instrument is to exercise a good temperature control over the sample. Another important variable is the light sensor (or detector) temperature. Typically the dark current (related to the signal noise) is related to the detector temperature. A control of this at constant temperature keeps the output noise within a given range resulting in better uniformity and reproducibility. The detector temperature for all measurements should be maintained within ±5° C. and more preferably within ±1° C. Typically when the detector is maintained at colder temperatures (e.g., 20 to 100° C. below the ambient temperature), the noise is significantly reduced resulting in superior signal to noise ratio. However, it is preferred to keep cooled detectors in sealed space or purged with dry gas to avoid any condensation of moisture. As an example, avalanche photodiodes may be used as detectors. These detectors are also available where they are integrated with a thermo-electric cooling plate from Advanced Photonix (Camarillo, Calif.) with part numbers as 118-70-74-591 and 197-70-74-591, etc. Alternatively one may procure light sensors such as UDT-020UV and UDT-050UV (from UDT Sensors Inc, Hawthorne, Calif.) and put them in close contact with thermoelectric plates such as those available from Jameco electronics (Belmont, Calif.) as TE chips 172030. When the sample is irradiated by a light source the temperature increases, and this increase also depends on the length of irradiation time. Thus it is desired that the irradiation time be controlled. One way of ensuring this is to irradiate the sample only for the duration for which the data on the light sensor is collected. This period is typically called the integration time and is usually less than a minute, typically in 1 to 5 seconds range. This temperature can be controlled by providing a shutter between the light source and the sample which is only opened by the microcontroller when the data is being collected. Another alternative may be an LED (light emitting source) which is powered or turned on during the integration time, as long as the LED lamps reach their steady state spectral emission within a fraction of a second of being powered (preferably in less than 1/10^{th} of the integration time). Another way is this LED to pulsate so that any thermal load is effectively dissipated. In very sensitive measurements with short integration times the main system controller can ensure that the thermoelectric plates are not powered during the short measurement time so that temperature fluctuations can be minimized.

Using light sources with low luminous energy output and detectors with high sensitivity, allows a better control over temperature of the sample and the detector due to lower amount of heating. As an example, since LEDs have low luminous output power, heating may not be an issue. LEDs may have peak output intensity between 340 and 390 nm with most preferred range being 360 to 380 nm. LED source LED380 from Ocean Optics (Dunedin, Fla.) has a light output power of 45 μW when coupled to an optical fiber cord of 600 μm. This is not too much power for significant temperature increase even if it lights a 2 ml sample in a cuvette continuously. A preferred power output ratio of the lamp to the solution volume should be less then 10 mW/ml for reduced thermal load, and a more preferred ratio is less then lmW/ml. Further, since the absorption for fluorescence is in a wide range at about 380 nm, it is best to use lamps with a spectral output in a tight range around this wavelength to reduce both the power consumption and the thermal load. An example of a preferred LED that has peak emission at 360 nm is L360-30M32L and another at 375 nm is L375-30K42L from Marubeni America Corporation (Santa Clara, Calif.). This can be coupled to an optical fiber or through lenses collimated into the sample. When such low powered lamps are used it is important to use filters with high transmission, and detectors with high sensitivity. The transmission of excitation filter (at the input before the light hits the sample) should be preferably in excess of 50%. An example of a narrow band filter that transmits between 350 and 400 nm is FF01-377/50 from Semrock (Buffalo, N.Y.). The transmission of the emission filter should also be preferably greater than 50%. An example of a narrow band filter that transmits between 460 and 488 nm is FF01-475/20 from Semrock. Both of these filters have transmission in excess of 90% at 360 to 380 nm and 465 to 485 nm respectively. Emission filter that transmit light between the wavelengths of 400 to 550 nm may be used, but those with transmissions greater than 50% anywhere between 450 and 500 nm are preferred. The most preferred filter has a peak transmission from about 475 to 480 nm with a bandwidth of lower than ±20 nm. An example of a sensitive photosensor is H5784 from Hamamatsu Corporation (Middlesex, N.J.) which has a peak sensitivity in excess of 3V/nW at 475 nm. The sensors preferred to be used with these LEDs should have sensitivities greater then 0.1 V/nW anywhere in the range of 460 to 515 nm.

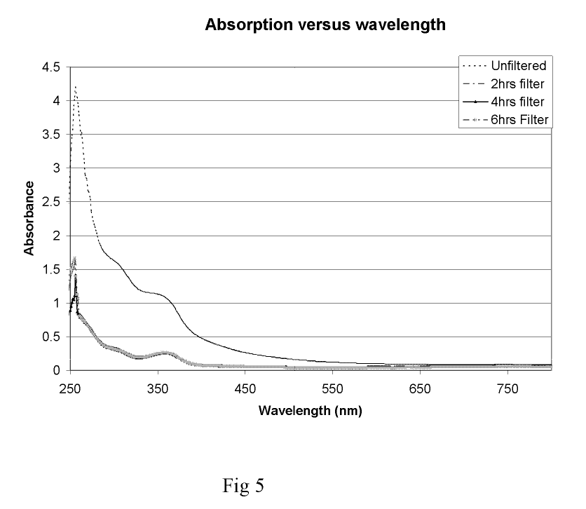

Further, a fluorimeter equipped to look at absorbance and fluorescence is most suited for this method. Absorbance is used to measure the yellowness of the solution to see if the results will be compromised due to the presence of excess iron or titanium. FIG. 5 shows an example of the spectra where the sample has beryllium and iron. This data is for soils as explained in Example 4, but it is applicable for other type of samples as well. If the samples are yellow, it is best to wait for some time so as to allow iron or other impurities to precipitate, so that the solutions can be filtered again (usually through a filter size of less than or equal to 2 microns, a preferred filter used had a pore size of 0.45 microns). The waiting period is typically between 30 minutes to 6 hours. Alternatively, the measurement solutions may be filtered sooner (smaller waiting times of less than 30 minutes) by filtering through a smaller pore size filter such as smaller than 0.25 microns, preferably about or less than 0.1 microns. As seen in FIG. 5, the absorption resulting in yellowness is quite broad and can be measured by measuring absorption or transmission in a wavelength range of 250 to 650 nm, preferably between 400 to 450 nm. The same lamp that is used for excitation may also be used for measuring the absorption.

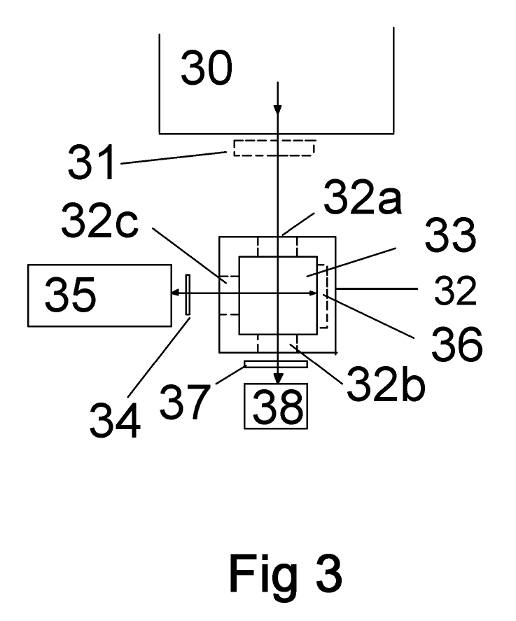

FIG. 3 shows a schematic diagram (top view) of a setup to measure both fluorescence and absorption using the same light source. The excitation light source 30 is shown along with excitation filter 31. The sample holder 32 has apertures 32a, 32b and 32c for the light to travel from or to the sample compartment 33. 34 is the emission filter for fluorescence and 35 is the fluorescence detector. 36 is a mirror or a reflector to reflect fluorescent light back into the detector 35 to increase the fluorescent intensity. 37 is the filter for absorption and 38 is the absorption (or transmission) detector. Filter 31 and 37 may have the same spectral characteristics, or the transmission window of 37 may be narrower than 31, but within the spectral range of 31. Filter 37 may be combined with an attenuating filter to adjust the intensity so that it is compatible with the detector 38. The sensitivity of the absorption detector need not be as high as the fluorescent detector. Examples of absorbance (or transmission) detectors are UDT-020D and UDT-055UV from OSI Optoelectronics Inc (Hawthorne, Calif.). If separate light sources are used, then for thermal reasons it is preferred to keep the light intensity for the absorption set-up low, thus LEDs with power output of 1 mW/ml of solution are preferred. The cuvette may remain in the same position so that both measurements can be made, or it may slide in the second position, or it may even be placed manually in the second position. Instead of absorption we may measure the color of the solution. As an example, a fiber optic color measurement sensor (RGB sensor) such as CZ-K series from Keyence Corporation of America (Woodcliff, N.J.) may be used. A specific example being CZ-60 sensor with CZK1 amplifier. The sensor head may be placed on one side of the sample and on the other side (180 degrees to it) of the sample a reflecting (such as a retro-reflector) or a white surface is placed.

One method to increase sensitivity is to have a strict temperature control during measurement as described earlier. Another way is to change chemistry so that more beryllium can be put in the “measurement solution”. As described in a preferred embodiment earlier which was taken from U.S. patent application Ser. No. 10/812,444, the volumetric ratio of the dissolution solution (comprising beryllium) to the detection solution (comprising dye) was 1:19. We found that ratios higher than 1:19 may be used to increase the detection limit of the method while keeping the other parameters constant. Increased ratios result in more beryllium in the detection solution thus increasing the sensitivity (lowering the detection of beryllium on the original media) of the method. Ratios higher than 1:12, e.g. such as 1:4 may be used to increase the beryllium content in the “measurement solution” by four times. One has to watch that the pH of the resulting “measurement solution” is still basic, preferably above 12 so that the fluorescence phenomena are not quenched. Further, the buffer capacity of the detection solution can be increased with more lysine. One has to be careful that increasing the concentration of a component does not increase the background signal during the measurement. Since there is more beryllium in the solution, it may also require more dye in the dye solution (or detection solution) to ensure that the upper-end of the range of beryllium detection range is not compromised (i.e., if this solution is used for determination of high levels of beryllium in the sample). This, when combined with the thermal modification described above, may allow detection limits to 0.0004 μg or lower per wipe or filter media. In a test method, all samples (solutions obtained after dissoluting beryllium or its compounds from the media) may be first analyzed using solution ratio of 1:19. Since only 0.1 ml of the 5 ml solution is analyzed in the above test (assuming that the sample is dissoluted in 5 ml of ammonium bifluoride solution), the remainder of the solution may be re-tested using the high sensitivity ratio of 1:4 for those samples which do not show presence of beryllium in the first analysis or those that show values of lower than 0.02 μg. Use of dilution modification to increase sensitivity has been disclosed in the patent application Ser. No. 11/152,620 and have been then subsequently published by Ashley et al, in Analytica Chimica Acta in 2007.

Some of the instruments which may be used for this purpose are available from Barnstead International (Dubuque, Iowa) models FM109515 and FM109535; from Turner Designs (Sunnyvale, Calif.) model numbers Aquaflor and TD700; from Optisciences (Tyngsboro, Mass.) model GFL1; and from Turner Biosystems (Sunnyvale, Calif.) model Modulus 9200-000.

Another, factor that leads to improvement in ultra-low detection is reducing the background fluorescence. The two primary sources are the material the cuvette is made out of and secondly the various components in the measurement solution. Table 1 shows how the background changes when different solutions and cuvettes are used. The background from the disposable cuvettes is quite high and the most significant contributor. For the simplicity of the test, cost and user convenience, these cuvettes are preferred. However, background fluorescence from the solution may be substantially reduced by simple changes to the materials used. Higher purity materials may be used that have low fluorescence.

Typically, the dye concentration using BBQS dye has been 63 μM. If there is 4 μg of beryllium on the media then this amounts to only 4 μM of beryllium in the solution. Since most of the regulations (such as Department of Energy's 850CFR regulation) call for action in a range of 0.2 to 4 μg of beryllium, thus, even if we are able to quantify to 12 μg of beryllium on the media, it is sufficient for most practical purposes. Thus one can use the lower dye concentration for all tests, typically lower than 50 μM to reduce the background.

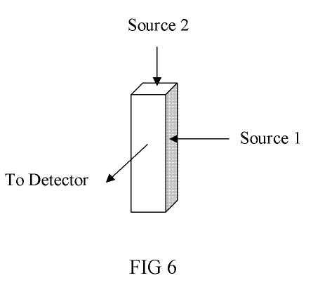

Most fluorometers to analyze cuvettes are typically configured with the excitation as shown in FIG. 6 by Source 1 (incoming excitation beam). In this case the cuvette walls are also excited by the incoming beam particularly if it is in UV range. For beryllium measurement it is in the UV, i.e., less than 400 nm. However, if the incoming beam is directed from the top into an open cuvette or directly into the solution (source 2), then the fluorescence from the walls is largely avoided. Thus a fluorometer to measure beryllium using this geometry is preferred, where the incoming beam is not passed through the container (or cuvette) wall before it impinges on the solution. One may also use a capped container with a small hole at the top to allow an LED or a fiber optic access, or may use a low fluorescence cover plate such as quartz to isolate optics from the chemicals. All these for the purpose of the invention are considered as open container.

TABLE 1
Background Noise (relative arbitrary units)

| | | | Detection | Detection solution | Detection solution |
|---|---|---|---|---|---|
| Type of | No solution, | | solution | with 1/10th dye | with normal dye |
| Cuvette | empty Cuvette | Water only | w/o dye | concentration | concentration |
| Quartz | 78 | 72 | 185 | 208 | 380 |
| Disposable | 250 | 755 | 990 | 1009 | 1030 |

For maximum effectiveness, one may combine changes in the instrument (including optics and light path configuration), dilution ratio and the dye concentration to get the most optimum solution and lowest detection. It is possible to detect beryllium in less than 0.1 ng on a media.

For analysis of naturally occurring beryllium in soils, one has to modify the method of dissolution and treatment of the measurement solution to get accurate results. It is important that the soils for analysis are prepared by milling, drying and sieving so that the particle size is in a certain range. Since the soil has to be dissoluted to extract beryllium, particle size will have an effect on the accuracy of results due to the kinetics of dissolution. It is preferred that the soils be sieved through a screen which has a mesh size of about 100 microns or smaller, with a preferred size of less then 50 microns. The dissolution time for soil is dependent on its chemical make-up, and may require longer dissolution times even when elevated temperatures are used. These times may be from 2 hours to 80 hours and may be shortened by simultaneous use of shaking and/or ultrasonic vibrations. Since, soils comprise of many elements including iron and titanium, this can lead to solutions (measurement solutions after mixing dissolution solution and the dye solution) which are slightly colored (yellow) or have higher absorption in the UV. These samples may have to sit for a while before these elements precipitate in the high pH solution and then may be re-filtered prior to use. This sitting period may be from about 30 minutes to six hours. The sitting time may also be dependent on the filter pore size. For example 0.45 micron pore size may require two hours or more and a 0.1 micron pore size may reduce the waiting period to less then 30 minutes. The fluorescence in soils may also be caused by the organic matter such as humic acid or others which may have phenol type structures. Thus it is desirable to include in a test protocol an additional process of heating to an elevated temperature, before the dissolution step. This treatment is to burn or destroy the organics to a point so that they do not fluoresce. Temperatures which are suitable for this treatment are in excess of 250 C, preferably in a range of 300 to 500 C for a duration of 15 minutes to about two hours. However, in this case one has to assess that no beryllium is lost due to the heating process.

Since this method requires considerable pipetting, thus it may become quite labor intensive. For automated system, one may use flow cells for measuring fluorescence, where solutions are automatically drawn from various solutions, individually mixing with a known quantity of the detection solution and analyzing as this mixture flows through a transparent tube (e.g. made out of quartz). The flow through cell needs to be automatically cleaned using a liquid and or gaseous media between different samples. The temperature of the tube is controlled for high reproducibility and low noise. The flow-through systems are available from Agilent (Palo Alto, Calif.) and from Perkin Elmer (Boston, Mass.). Automation may also be achieved by using an auto-sampler where the standards and the unknown samples are pre-arranged in a specific fashion in a tray and cuvettes are prepared for measuring these rather than the flow cells. The auto-sampler picks or routes these cuvettes, e.g., one at a time in the fluorometer and measures these.

A semi-automated system with multiple modules may be used to decrease the labor and be able to match the throughput of the system at various stages according to their requirements and budgets. As an example a module for dissolution may comprise of loading a cassette with tubes containing samples (soil, wipes or filters). The module is programmed to automatically pipette accurate amounts of the dissolution solution in each of these and then to heat these tubes for the specified time, e.g., in a block heater or an oven. After which the samples are cooled and then an alarm is sounded so that the samples can be manually transferred to the second module for preparation of measurement samples in cuvettes. The samples in the second module are filtered and an aliquot drawn and added to the detection solution into individual cuvettes or microtitrator plate chambers. As an added option to this module, one may hold the cuvettes for a period of time (30 minutes to 4 or more hours) so that metals such as iron and titanium precipitate and can be removed through filtration. As described above, this time may be reduced by using finer pore filters. At the end of this step an alarm sounds and the cuvettes with twice filtered solutions or as the case may be, are manually transferred to the measurement or the third module. In the measurement module the sample is measured for absorbance to see if it is still yellow due to the presence of titanium, iron, etc, and then measured for fluorescence. The wavelength in which the yellowness or the presence of the metals is measured is between 250 and 500 nm, and more preferable between 350 and 450 nm. This measurement may be done at a single wavelength. The samples that are still yellow may be isolated for manual handling later or through an automatic procedure. In a complete automated mode all three modules may be integrated with robotic transfers including sample tracking which may utilize the bar code readers.

### Example 1

### Effect of Temperature

A fluorometer from Barnstead International (model FM109515) was used in this experiment. For excitation a narrow band filter (NB360) and for emission a narrow band filter (NB460) were used, both of these supplied by the instrument manufacturer. Detection solution was made by using 1.8 liters of deionized water (18 Mohms), 19.51 g of lysinemonohydrochloride, 1.99 g of EDTA disodium dihydrate, 0.0367 g of HBQS and then titrating this with a solution of 2.5N sodium hydroxide to a final pH of 12.85. 1.9 ml of the detection solution was poured in a fluorescent plastic cuvette. 0.1 ml of ammonium bifluoride solution comprising beryllium was added to the cuvette. Four different concentrations of beryllium solutions were prepared by adding 0.1 ml of 0, 2, 5 and loppm standards. These were used to calibrate the fluorometer. The calibration was a straight line with a correlation coefficient of 0.99. The standard with 5 ppm of beryllium was re-measured for fluorescence while its temperature was measured. The change in temperature occurred by leaving the sample in the fluorometer for an extended period of time and also placing the fluorometer in an area where the airflow was restricted. Thus the heat was produced by the illumination lamp. FIG. 4 shows the fluorescence value measured in the fluorometer and its change in temperature. When the solution was cooled to the original temperature the fluorescence went back to the original value.

### Example 2

### The Effect of the Extraction (Dissolution) Solution on the pH of the Detection Solution

A dissolution solution with 1% ammonium bifluoride and a detector solution were made as described in example 1. These solutions were mixed in different ratios and their pH measured. The data show that a ratio of 1:4 (dissolution solution to detection solution) still resulted in a pH in excess of 12.

| Dissolution | Detection | Volumetric ratio of “Dissolution | |
|---|---|---|---|
| solution (ml) | solution (ml) | solution:Detection solution” | pH |
| 0.1 | 1.9 | 1:19 | 12.46 |
| 0.4 | 1.6 | 1:4 | 12.16 |
| 0.5 | 1.5 | 1:3 | 11.39 |
| 1.0 | 1.0 | 1:1 | 8.55 |

### Example 3

### Measurement of Beryllium

### Increased Detection Sensitivity

A series of calibration solutions were prepared as shown in the table below.

| | Final concentration | |
|---|---|---|
| | of beryllium (ppb) | Corresponding amount |
| Preparation of | in calibration | of beryllium in the |
| Standard Solutions | standard solutions | media* |
| 0.4 ml of 0 ppb | 0.0 | Corresponds to |
| standard + 1.6 ml of | | 0.00 μg Be on media |
| detection solution | | |
| 0.4 ml of 1 ppb | 0.2 | Corresponds to |
| standard + 1.6 ml of | | 0.005 μg Be on media |
| detection solution | | |
| 0.4 ml of 4 ppb | 0.8 | Corresponds to |
| standard + 1.6 ml of | | 0.02 μg Be on media |
| detection solution | | |
| 0.4 ml of 20 ppb | 4.0 | Corresponds to |
| standard + 1.6 ml of | | 0.1 μg Be on media |
| detection solution | | |
| 0.4 ml of 80 ppb | 16.0 | Corresponds to |
| standard + 1.6 ml of | | 0.4 μg Be on media |
| detection solution | | |

These correspond to the 1:4 dissolution solution to the detection solution ratio as discussed in Example 2. A series of filter papers were spiked with various amounts of beryllium oxide from slurries and analyzed. The filter papers Whatman 541 (Ashless, 4.7 cm in diameter) and mixed cellulose ester (MCE) filters that were 0.8 μm pore size and 3.7 cm in diameter were obtained from Fisher Scientific (Pittsburgh, Pa.). The analysis procedure comprised of dissoluting the spiked filters in 1% ammonium bifluoride aqueous solution at 80° C. for 30 minutes. The solutions were then cooled and filtered through 0.45 μm pore size nylon filters. Then 0.4 ml of these solutions were mixed with 1.6 ml of the detection solution (see details of detection solution in Example 1) and measured on three different fluorometers i.e., Turner Quantech model FM109515 (Barnstead, Dubuque, Iowa), Ocean Optics S2000FL (Dunedin, Fla.) and Spex Fluorolog 2 (Horiba Jobin Yvon, Edison, N.J.). The excitation wavelength was between 360 and 380 nm and the measurement wavelength for the turner instrument was 460 nm and 475 for the others. The results from these are shown in the table below, along with standard deviation (SD) and relative standard deviation (RSD).

Analysis results from MCE and Whatman 541 filters
spiked with ultra-low levels of beryllium.

| Spike level, μg Be (n = 6) | Mean (±SD), μg Be | RSD (%) |
|---|---|---|
| MCE Filters | | |
| Blank | N.D.¹ | — |
| 0.002 | 0.0023 (±0.00030) | 13 |
| 0.005 | 0.0052 (±0.00012) | 2.3 |
| 0.020 | 0.0210 (±0.00055) | 2.6 |
| 0.050 | 0.0504 (±0.0014) | 2.8 |
| Whatman 541 Filters | | |
| Blank | N.D. | — |
| 0.002 | 0.0025 (±0.00048) | 19 |
| 0.005 | 0.0056 (±0.00035) | 6.3 |
| 0.020 | 0.0209 (±0.00049) | 2.3 |
| 0.050 | 0.0507 (±0.00013) | 2.6 |

Data are pooled from three different laboratories (two samples from each, total samples 6) using three different fluorometers.
¹None detected (<MDL)

The method detection limit (MDL) was estimated by measuring a minimum of ten clean (unspiked) filters, and reporting the MDL as three times the standard deviation of repeat media blank measurements. These results are shown in the table below. These results are only for the specific dilution ratio of 1:4 as discussed above.

| | MDL, | MDL, |
|---|---|---|
| Fluorometer | MCE filters | Whatman 541 filters |
| Turner Quantech | 0.00075 μg | 0.00078 μg |
| Ocean Optics S2000-FL | 0.0015 μg | 0.0016 μg |
| Spex Fluorolog2 | 0.0019 μg | 0.0021 μg |

### Example 4

### Analysis of Soils

Beryllium Extraction from NIST, Standard Reference Material (SRM) 2710, Montana Soil:

0.500 grams of Montana soil obtained from National Institute of Standards and Technology (NIST, Gaithersburg, Md.) SRM 2710 was weighed out on a balance and transferred to a 60 ml Nalgene polypropylene bottle with a flat bottom and fitted with a sealing cap. To this was added using a 25 ml graduated pipette 50 ml of 5 wt % ammonium bifluoride in deionized water for dissolution of beryllium. The mixture was hand shaken and placed in a forced air convection oven, preheated to 80° C., for 16 hours. Upon removal from oven the dissolution solution was left standing at room temperature for 30 minutes to cool.

### Addition of Dye for Fluorescence Measurement:

5 ml of the above dissolution solution was placed in a syringe and filtered through a 0.45 micron nylon filter (Millipore Millex 13 mm) into a 10 ml falcon tube. 0.1 ml of this solution was removed using an Eppendorf Research 100 μL fixed pipette and placed in a 4.5 ml low fluorescence acrylic cuvette. To this cuvette was also added 1.9 ml of detection solution (see example 1 for the description of the detection solution) containing a fluorescent dye using a Eppendorf Reference variable (2500 to 500 μL) pipette. The cuvette was capped, hand shaken and stored in a sealed amber colored jar.

### Measurement of Fluorescence:

A Turner Quantech FM 109515 Fluorometer fitted with a 360 nm excitation filter and a 460 nm emission filter was used to measure fluorescence. All measurements were performed under ambient atmosphere at room temperature. The fluorometer was calibrated using 0, 10, 40 and 200 ppb Beryllium Standards from SPEX CertiPrep (Metuchen N.J.) by placing 0.1 ml of each standard in a cuvette and adding 1.9 ml of the detection solution. The detection solution was prepared as given in Example 1. The final concentration of beryllium in the cuvettes was 0, 0.5, 2.0 and 10 ppb respectively. The standards were measured sequentially, which gave a reference plot with a correlation coefficient of one between the concentration of beryllium and the fluorescent intensity. The soil extract in the cuvette, prepared as described above, was measured and the fluorescence value compared against the calibration curve, which gave a value of 1.45 μg of beryllium per 0.5 g soil sample. This solution was yellow in color indicating the presence of iron which in high amounts interferes with the beryllium fluorescence measurement. To eliminate this problem the sample in the cuvette was left standing at room temperature for 24 hours at which point the iron precipitates out. The solution was again filtered through a 0.45 micron nylon filter into a new cuvette. The filtered solution was colorless. It was then re-measured for fluorescence and gave a value of 1.57 μg of beryllium per 0.5 g soil sample or a mass fraction of beryllium of 3.13 mg/kg.

### Protocol Development

In order to develop the above protocol several experiments were done. In initial experiments, conical-end shaped centrifuge tubes were used for dissolution (or extraction). As the results show below, flat bottom tubes or bottles are preferred as the extraction is more complete. For example the 15 ml capacity conical end shaped tubes using 0.1 g of soil and 10 ml of 5% ammonium bifluoride solution resulted in measuring 2.28 mg of beryllium in one kg of soil. Using the same method while using flat bottom bottles for dissolution, other NIST standards were also analyzed, these were SRM 1944 (NY/NJ Sediment) and SRM 2702 (Inorganic Marine sediment). The results obtained are compared to those reported by NIST which were determined by inductively coupled plasma (ICP)-Atomic Emission Spectroscopy (AES).

| | Results from fluorescence | Results Reported by NIST |
|---|---|---|
| Sample Type | Beryllium, mg/kg | Beryllium, mg/kg |
| SRM 1944 | 2.07 | 1.6 |
| SRM 2702 | 2.83 | 3 |

Having established that it was important to use flat bottom tubes or bottles a systematic study to develop a suitable protocol was undertaken. For this, NIST SRM 2702 (Marine sediment) was used. The soil sample was fixed at 0.5 g and a dissolution solution of 50 ml of 5% ABF was used. The dissolution temperature was 80° C. with a dissolution time of 16 hours. The measurement solutions were made by adding 1.9 ml the dye solution to 0.1 ml of filtered dissolution solution as described above. The solution was measured immediately and also after letting stand for 2, 4, 6, 24 hours and filtering through 0.45 micron pore filters. The amount of beryllium calculated from the results was 1.06, 3.31, 3.33, 3.36 and 3.33 mg/kg of soil respectively. This shows that a waiting period of 2 hours is sufficient for this sample. Absorbance spectra of the measurement solution after each of these time periods (and re-filtering) are shown in FIG. 5.
In another series of experiments a number of soils were evaluated using 3% ABF solution. The dissolution time at 90 C was 40 hours. In each case 0.5 g of sample was used along with 50 ml of dissolution solution. The beryllium values “value provided” was the one that came with the certificates of these standard materials, excepting for SRM2710 which was taken from the literature. More details are provided in the reference from Agrawal et. al. in Environmental Science and Technology publication.

TABLE
Beryllium concentrations established by fluorescence using 0.5 g of
soil dissolved in 50 ml of 3% (w/w) ABF for 40 hours at 90° C.

| | Particle Size | Beryllium (Value | Beryllium (From |
|---|---|---|---|
| Reference Material | Information | provided) (mg/kg) | Fluoresence (mg/Kg) |
| NIST (National institute of | Passed through | 3 | 3.50 |
| Standards and Technology, | 70 μm screen | | |
| Gaithersburg, MD) SRM 2702, | | | |
| Marine Sediment | | | |
| NIST SRM 2710, Montana Soil | Passed through | 2.5 | 3.35 |
| | 74 μm screen | | |
| NIST SRM 1944, NY/NJ | Passed through | 1.6 | 2.37 |
| Waterway Sediment | 250 to 61 μm | | |
| | screens | | |
| NIST SRM 1633a, Coal Fly Ash | Less than 88 μm | 12.1 | 12.9 |
| GSJ (Gelologoical Survey of | Median 6.06 μm | 2.05 | 2.11 |
| Japan, Japan) JA-2, Andesite | | | |
| GSJ JR-3, Rhyolite | Median 4.57 μm | 7.6 | 7.1 |
| GSJ JB-2, Basalt volcanic rock | Median 5.41 μm | 0.27 | 0.31 |
| CCRMP (Canadian certified | Passed through | 22 | 21.4 |
| Materials Project, Ontario, | 74 μm screen | | |
| Canada) SY2 Syenite | | | |
| CCRMP Till-1 Soil | Passed through | 2.4 | 2.53 |
| | 74 μm screen | | |

While this invention has been described as having preferred sequences, ranges, steps, materials, structures, features, and/or designs, it is understood that it is capable of further modifications, uses and/or adaptations of the invention following in general the principle of the invention, and including such departures from the present disclosure as those come within the known or customary practice in the art to which the invention pertains, and as may be applied to the central features hereinbefore set forth, and fall within the scope of the invention and of the limits of the appended claims.

What is claimed is:

1. A method of determining the presence or amount of beryllium or a beryllium compound in air, comprising:
drawing a sample of the air containing particulates of beryllium through a liquid medium,
trapping the particulates and analyzing the said liquid medium for beryllium.

2. The method of claim 1, wherein the liquid medium comprises dissolution solution for beryllium.

3. The method of claim 2, wherein the dissolution solution comprises ammonium bifluoride.

4. The method of claim 1, wherein the detection comprises a step of preparing a “measurement solution” by mixing a portion of the said liquid medium comprising beryllium with a solution comprising fluorescent indicator and determining the presence or amount of beryllium compound within said air sample by measuring fluorescence in the “measurement solution” from said fluorescence indicator.

5. The method of claim 4, where the fluorescent indicator comprises, 10-hydroxybenzo[h]quinoline-7-sulfonate.

6. A method of determining the presence or amount of beryllium or a beryllium compound in a soil sample, comprising:
admixing a sample suspected of containing beryllium or a beryllium compound with a dissolution solution for sufficient time at a temperature in excess of 50° C., whereby beryllium or a beryllium compound within said sample is dissolved;
preparing a “measurement solution” by mixing a portion from said admixture with a detection solution comprising a fluorescent indicator capable of binding beryllium or a beryllium compound to the fluorescent indicator; determining the presence or amount of beryllium or a beryllium compound within said sample by measuring fluorescence from said “measurement solution”.

7. A method of determining the presence or amount of beryllium or a beryllium compound in a soil sample as in claim 6 where the dissolution solution comprises one of ammonium bifluoride, concentrated nitric acid, concentrated hydrochloric acid and concentrated sulfuric acid.

8. A method of determining the presence or amount of beryllium or a beryllium compound in a soil sample as in claim 7, where in the dissolution solution, the ratio of ammonium bifluoride to soil is greater than 1:1 by weight.

9. A method of determining the presence or amount of beryllium or a beryllium compound in a soil sample as in claim 7 where the dissolution solution comprises of 1% to 5% by weight of ammonium bifluoride in water.

10. A method of determining the presence or amount of beryllium or a beryllium compound in a soil sample as in claim 6, where the dissolution is carried out by heating the solution between a range of 50 to 100° C.

11. A method for determining the presence or amount of beryllium or a beryllium compound, wherein the detection comprises a step of preparing a “measurement solution” by mixing a portion of sample comprising beryllium in with a solution comprising fluorescent indicator and determining the presence or amount of beryllium compound within said sample by measuring fluorescence wherein the excitation beam impinges on the solution without passing through the walls of the container comprising the “measurement solution”.

12. A method for determining the presence or amount of beryllium or a beryllium compound as in claim 11, where additional information is obtained by measuring absorption or the color of the “measurement solution”.
